(19) United States
(12) Patent Application Publication
(10) Pub. No.: US 2024/0390269 A1
(43) Pub. Date: Nov. 28, 2024

(54) USE OF PERILLA IN THE MANUFACTURE OF MEDICAMENT FOR INHIBITION OF COVID-19 VIRUS

(71) Applicants: YING-LING HUANG, KAOHSIUNG CITY (TW); CHENG-CHEUNG CHEN, TAIPEI CITY (TW); CHANGAN NANO APPLIED BIOTECHNOLOGY CO., LTD., TAINAN CITY (TW)

(72) Inventor: YING-LING HUANG, KAOHSIUNG CITY (TW)

(21) Appl. No.: 18/324,296

(22) Filed: May 26, 2023

(51) Int. Cl. A61K 9/00; A61K 36/15; A61K 36/232; A61K 36/236; A61K 36/534; A61K 36/535; A61K 36/752

(52) U.S. Cl. CPC A61K 9/006; A61K 36/15; A61K 36/232; A61K 36/236; A61K 36/534; A61K 36/535; A61K 36/752

(57) ABSTRACT

A use of perilla in the manufacture of a medicament for inhibition of Coronavirus disease 2019 (COVID-19) virus is provided. Perilla is treatment by ultrafine pulverization and wall-breaking to obtain perilla micropowder with a particles size of 6000 mesh. The perilla micropowder is used in the manufacture of an inhibitor of COVID-19 virus (SARS-COV-2,Omincron variant) and a medicament for treatment, prevention, or relief of diseases caused by COVID-19 virus (SARS-COV-2,Omincron variant).

### BACKGROUND OF THE INVENTION

### Field of the Invention

The present invention relates to a medicament for inhibition of COVID-19 virus, especially to a use of perilla in the manufacture of a medicament for inhibition of COVID-19 virus and diseases caused.

### Description of Related Art

In 2019, a novel coronavirus emerged. The disease caused by the unidentified coronavirus was officially names as Coronavirus Disease-2019 (COVID-19) by world health organization (WHO) in 2020. International Committee on Taxonomy of Viruses (ICTV) announced “severe acute respiratory syndrome coronavirus 2 (SARS-COV-2)” as the name of the new virus. SARS-COV-2 is a betacoronavirus, a genus in Coronavirinae. Coronavirinae (CoV) are a group of enveloped viruses with a positive-sense single-stranded RNA genome, rounded-shaped. The CoV is round-shaped with spikes projecting from surface of the virion and delivering the virion the appearance of a solar corona under scanning electron microscopy (SEM), prompting the name coronaviruses.

According to research of Taiwanese ministry of health and welfare, droplets with different sizes and containing viruses are released when people infected with SARS-COV-2 is breathing, talking, singing exercising coughing or sneezing. Droplet nuclei with smaller diameter suspend in the air from several minutes to several hours while droplets with larger diameter fall to the ground or various object surfaces quickly. Thus the virus can be transmitted through inhalation, direct contact or indirect contact. Moreover, a confirmed patient may be contagious 2 days before the onset of illness. After the onset of illness, viruses are still generated within and released from the respiratory tract of the patients. That's why the epidemic has spread rapidly across the world.

Currently no specific treatment is available for patients with COVID-19 virus so that current treatment is limited to supportive care clinically. Thus there is an urgent need to develop medications which inhibit coronavirus COVID-19 effectively

### SUMMARY OF THE INVENTION

Therefore, it is a primary object of the present invention to provide perilla micropowder having a particle size of 6000 mesh used in the manufacture of an inhibitor and a medicament for inhibition of COVID-19 virus and the perilla micropowder is produced by ultrafine pulverization and wall-breaking of perilla.

In order to achieve the above object, perilla micropowder used in the manufacture of an inhibitor of COVID-19 virus (SARS-COV-2,Omincron variant) and a medicament for treatment, prevention, or relief of diseases caused by COVID-19 virus (SARS-COV-2,Omincron variant) is provided. The perilla micropowder is produced by ultrafine pulverization and wall-breaking of perilla and a diameter of the perilla micropowder is 6000 mesh.

In the use of perilla in the manufacture of a medicament for inhibition of COVID-19 virus, the medicament is oral spray powder.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a scanning electron microscopy (SEM) image (magnification ×5000) showing particles size of perilla micropowder of an embodiment according to the present invention;

FIG. 2 is another scanning electron microscopy (SEM) image (magnification ×5000) showing particles size of perilla micropowder of an embodiment according to the present invention;

FIG. 3 is a further scanning electron microscopy (SEM) image (magnification ×5000) showing particles size of perilla micropowder of an embodiment according to the present invention;

FIG. 4 is a test result chart of the inhibitory effect that the perilla micropowder inhibits the COVID-19 virus (SARS-COV-2,Omincron variant) by virus plaque assay.

### DETAILED DESCRIPTION OF THE PREFERRED EMBODIMENT

In order to learn technical content, purposes and functions of the present invention more clearly and completely, please refer to the following detailed descriptions with the figures and reference signs.

An antiviral medicament that inhibits coronavirus includes perilla micropowder which is produced by ultrafine pulverization and wall-breaking of perilla using a dry-type pulverizer and a particle size of the micropowder is 6000 mesh. The perilla micropowder is used in the manufacture of an inhibitor of COVID-19 virus (SARS-COV-2,Omincron variant) and a medicament for treatment, prevention, or relief of diseases caused by infection of COVID-19 virus (SARS-COV-2,Omincron variant). The inhibitor and the medicament can be oral spray powder.

The preparation of the perilla micropowder includes the steps of washing and drying whole perilla composed of roots, stems, and leaves and then performing ultrafine pulverization and wall-breaking processes. A particle size of the perilla micropowder obtained is 6000 mesh.

Super-fine crushing and wall breaking of the perilla is performed by an airflow pulverizer so that the prilla is super-micronized into micron-scale particles. When raw material of the dry perilla is placed into a grinding chamber of the airflow pulverizer, super-high-speed air flows are sprayed from a plurality of airflow nozzles mounted in the crushing chamber. Due to impact of super-high-speed air flows and collision among the dry perilla, the dry perilla is pulverized into perilla micropowder with a particle size of 6000 mesh and physical wall-breaking of the perilla is also achieved. The super pulverization and wall-breaking of perilla can be achieved by a plurality of airflow pulverizers and an apparatus for fining powders revealed in US Pat. No. 7,472,852 B2 is preferred.

Since the particle size of the perilla micropowder is micron-scale (6000 mesh) and the walls of cells of the perilla micropowder have been broken, human bodies can absorb effective substances contained in perilla completely.

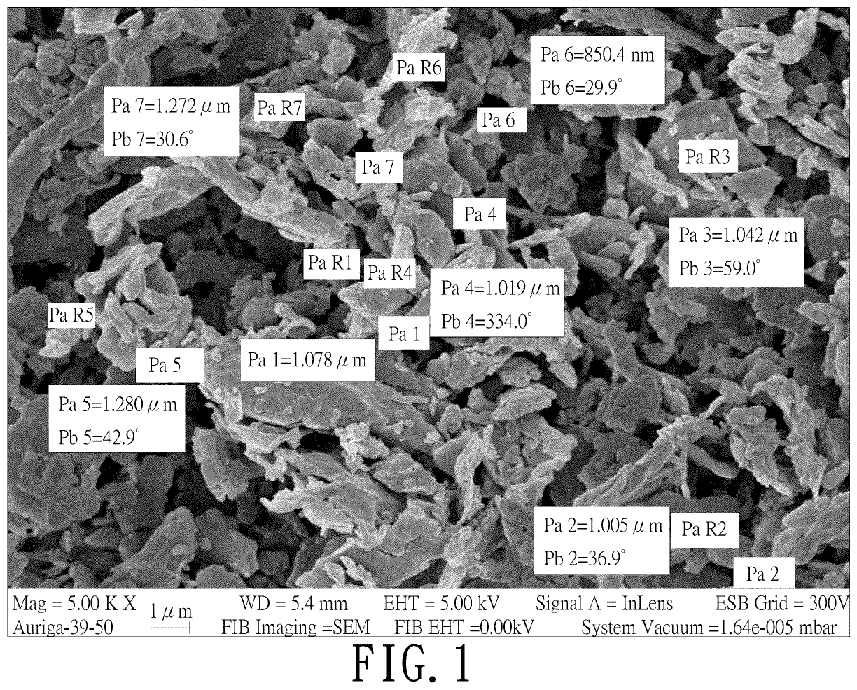

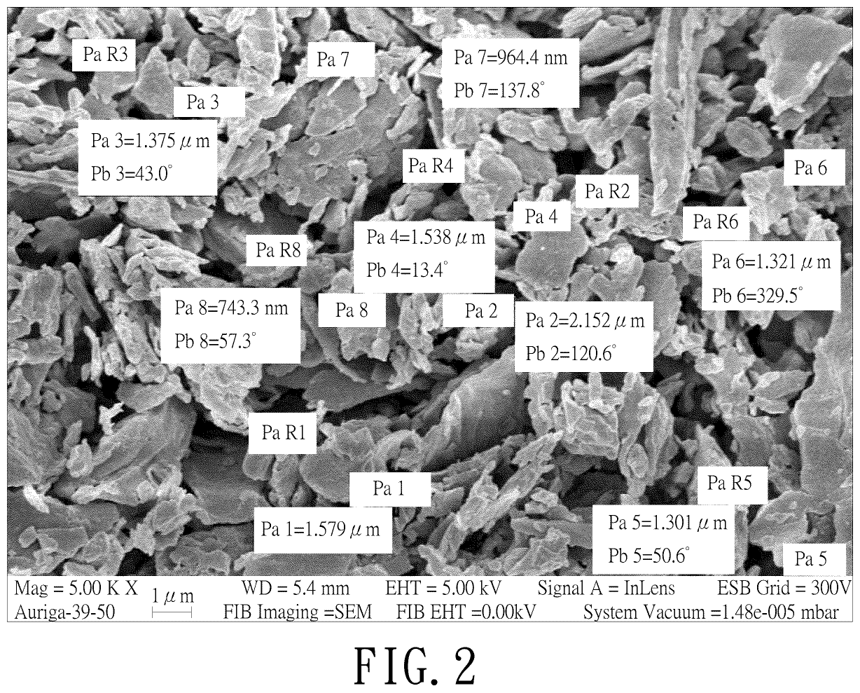

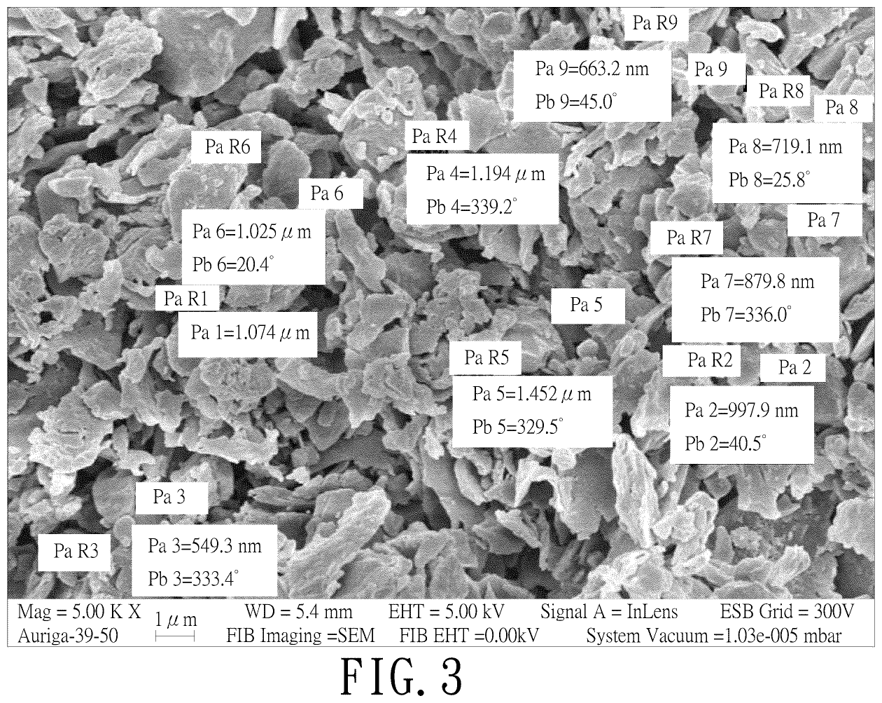

Refer to FIG. 1-3, the perilla micropowder obtained after ultrafine pulverization and wall-breaking are magnified 5000 times and analyzed in a scanning electron microscopy in order to detect the particle size (diameter). The particle size of the perilla micropowder is smaller than 8 micrometers (μm) and a diameter D50 (particle size distribution) is no more than 2-5 μm (≤2-5 μm).

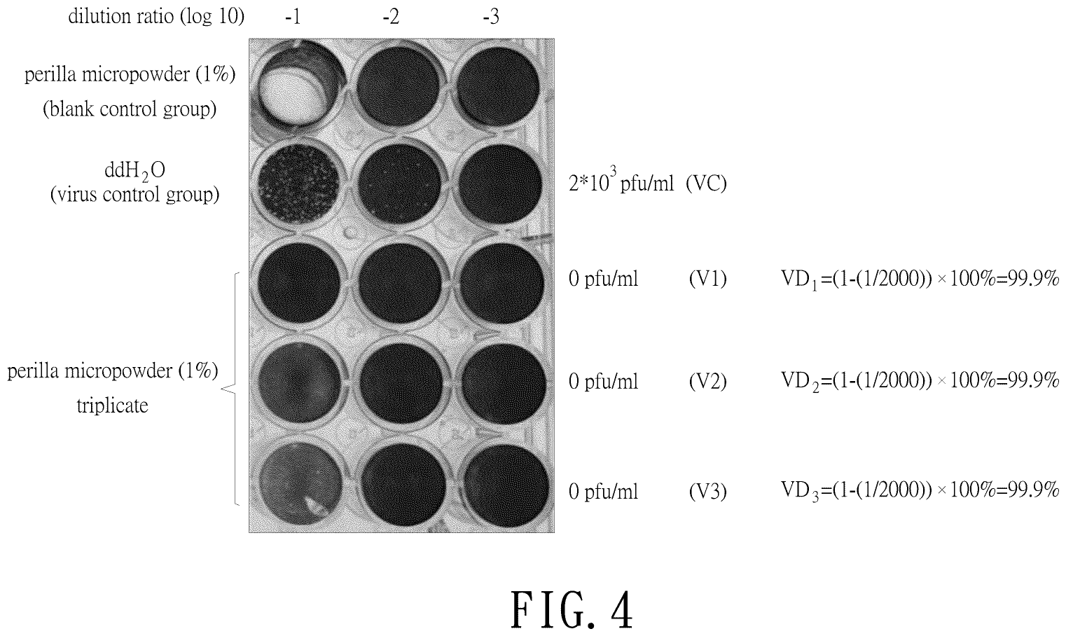

Moreover, use plaque assays to test whether the perilla micropowder obtained after ultrafine pulverization and wall-breaking can inhibit COVID-19 virus (SARS-COV-2, Omincron variant) or not. Take 1 wt % of the perilla micropowder obtained and test cells which are Vero C1008 [Vero 76,clone E6, Vero E6] (CRL-1586) to react for 30 seconds. The test results shown in FIG. 4 indicate that the micronized perilla micropowder can inhibit 99.9% COVID-19 virus (SARS-COV-2,Omincron variant).

The wall-broken perilla micropowder with the particle size of 6000 mesh is mixed with an oil-water double layer liquid in a ratio of 1:100 (wt %) to form a liquid-form perilla composition. In one embodiment, the liquid-form perilla composition is placed in a spray bottle and used as a spray.

In one embodiment, the oil-water double layer liquid comprises a lower layer liquid and an upper layer liquid, the ratio between the lower layer liquid and the upper layer liquid is 28:4 (wt %), and the specific gravity of the upper layer liquid is smaller than that of the lower layer liquid, so that the upper layer liquid is suspended on the lower layer when the liquid-form perilla composition is in a static state.

In one embodiment, the lower layer liquid is a liquid containing a surfactant, and when the liquid-form perilla composition is placed in a container and shaken, the upper layer liquid and the lower layer liquid can be mixed for a short time.

Further, in one embodiment, the upper layer liquid comprises an extract of oil-extracted licorice and angelica and herbal essential oil, the ratio of the extract of oil-extracted licorice and angelica to herbal essential oil is 1:1 (wt %), and the composition of the herbal essential oil comprises Cedarwood, Nepeta, Peppermint, Ylang-ylang and Neroli.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

What is claimed is:

1. A method for treatment, prevention, or relief of diseases caused by Coronavirus disease 2019 virus (COVID-19 virus) (SARS-COV-2,Omincron variant), comprising administering a medicament containing perilla micropowder to a subject in need; wherein the perilla micropowder inhibits the COVID-19 virus (SARS-COV-2,Omincron variant).

2. The method as claimed in claim 1, wherein the medicament is oral spray powder.

3. The method as claimed in claim 2, wherein the perilla micropowder is mixed with an oil-water double layer liquid in a ratio of 1:100 (wt %) to form a liquid-form perilla composition; the oil-water double layer liquid comprises a lower layer liquid and an upper layer liquid, the ratio between the lower layer liquid and the upper layer liquid is 28:4 (wt %), and the specific gravity of the upper layer liquid is smaller than that of the lower layer liquid.

4. The method as claimed in claim 3, wherein the lower layer liquid is a liquid containing a surfactant.

5. The method as claimed in claim 4, wherein the upper layer liquid comprises an extract of oil-extracted licorice and angelica and herbal essential oil, the ratio of the extract of oil-extracted licorice and angelica to herbal essential oil is 1:1 (wt %), and the composition of the herbal essential oil comprises Cedarwood, Nepeta, Peppermint, Ylang-ylang and Neroli.

6. The method as claimed in claim 1, wherein the perilla micropowder is mixed with an oil-water double layer liquid in a ratio of 1:100 (wt %) to form a liquid-form perilla composition; the oil-water double layer liquid comprises a lower layer liquid and an upper layer liquid, the ratio between the lower layer liquid and the upper layer liquid is 28:4 (wt %), and the specific gravity of the upper layer liquid is smaller than that of the lower layer liquid.

7. The method as claimed in claim 6, wherein the lower layer liquid is a liquid containing a surfactant.

8. The method as claimed in claim 7, wherein the upper layer liquid comprises an extract of oil-extracted licorice and angelica and herbal essential oil, the ratio of the extract of oil-extracted licorice and angelica to herbal essential oil is 1:1 (wt %), and the composition of the herbal essential oil comprises Cedarwood, Nepeta, Peppermint, Ylang-ylang and Neroli.

9. A method for inhibition of Coronavirus disease 2019 virus (COVID-19 virus) (SARS-COV-2,Omincron variant), comprising administering an inhibitor containing perilla micropowder to a subject in need; wherein the perilla micropowder inhibits the COVID-19 virus (SARS-COV-2,Omincron variant).

10. The method as claimed in claim 9, wherein the inhibitor is oral spray powder.
